(19) United States
(12) Patent Application Publication
(10) Pub. No.: US 2019/0087517 A1
(43) Pub. Date: Mar. 21, 2019

(54) FATIGUE LIFE PREDICTION METHOD AND DEVICE OF CONCRETE BASED ON WEIBULL FUNCTION AND MAXIMUM FATIGUE DEFORMATION

(71) Applicant: ZHEJIANG UNIVERSITY, Zhejiang (CN)

(72) Inventors: Shilang XU, Zhejiang (CN); Botao HUANG, Zhejiang (CN); Qinghua LI, Zhejiang (CN)

(21) Appl. No.: 15/871,288

(22) Filed: Jan. 15, 2018

(30) Foreign Application Priority Data
Sep. 19, 2017 (CN) 201710856490.9

(51) Int. Cl. G06F 17/50; G06F 17/11; G01N 3/32; G01N 33/38

(52) U.S. Cl. CPC G06F 17/5009; G06F 17/11; G06F 2217/16; G01N 33/383; G01N 3/32

(57) ABSTRACT

The present invention discloses a fatigue life prediction method and device of concrete based on Weibull function and maximum fatigue deformation. With the continuous development of modern civil engineering, the fatigue performance of concrete materials has become one of the focuses of concern. The accurate prediction of fatigue life of concrete has become an important issue in the field of engineering construction. The method and device provided in the invention can be used to predict the life of concrete and fatigue deformation evolution law of concrete under the fatigue loads, having the advantages of concise steps, simpleness to use and high accuracy, etc. During the use, it can greatly reduce the computations, and only two fatigue parameters of the number of fatigue load cycles n and the maximum fatigue deformation εₛ of the n^{th} cycle need to be measured, which simplifies the monitoring equipment. The model can provide an important technical support for engineering design, construction, monitoring and maintenance.

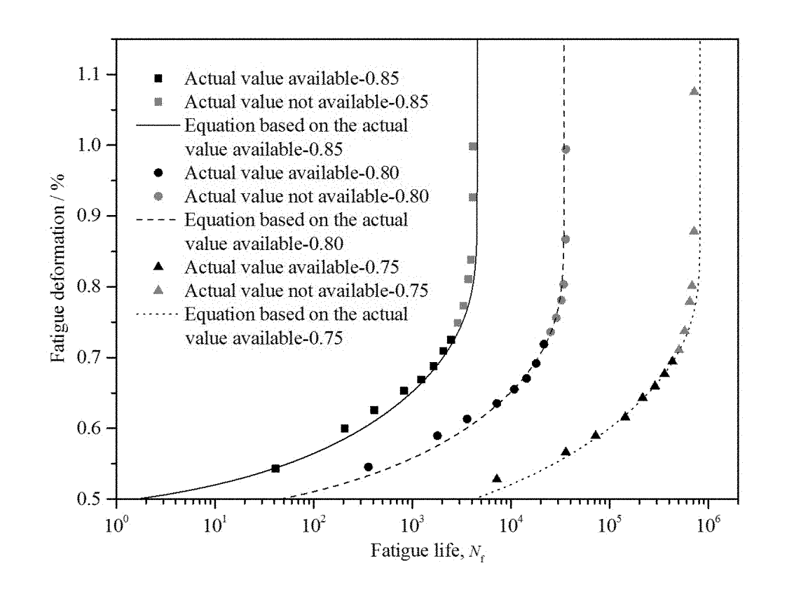

### FIELD OF THE INVENTION

The present invention relates to a fatigue life prediction technology of concrete.

### BACKGROUND

Since the advent of Portland cement in the 19^{th} century, concrete has been widely used in such fields as transportation, construction, water conservancy and marine engineering. It is the material used most widely in the engineering construction. In the early 20^{th} century, with the construction of reinforced concrete bridges, the related researches on the fatigue performance of concrete materials are gradually carried out. Since the 21^{st} century, with the construction of large-scale infrastructures such as highways, high-speed railways, super high-rise buildings, special high dams, cross-sea bridges and offshore platforms, concrete structures are faced with more complicated and harsh service conditions such as cyclic loads and alternating environments, etc. In addition, with the further development of the theory of concrete structure design and the popularization and application of high-strength concrete, the stress level of concrete is gradually increased during the service of the structure, which makes fatigue failure of concrete more likely. Therefore, in the continuous development of modern civil engineering, the fatigue performance of concrete materials has become one of the focuses of concern. How to accurately predict the fatigue life of concrete becomes an important issue in engineering design, construction, monitoring and maintenance. The existing characterization of fatigue performance and fatigue life prediction of concrete materials are mainly based on the evolution of materials' fatigue damage. Researchers have developed a series of fatigue models that establish the relationship of fatigue damage primarily through the attenuation of materials' elastic modulus and based on which, establish complex fatigue performance characterization and life prediction models. Existing models usually need to include many parameters such as fatigue strain, fatigue stress, elastic modulus and materials' fitting parameters. The model is complicated and generally needs to be iteratively calculated. Thus, it is difficult to popularize and apply it in engineering construction. Therefore, it is very urgent to propose a fatigue life prediction method and device of concrete with concise steps, simpleness to use and high precision, which can provide important technical support for engineering design, construction, monitoring and maintenance.

### SUMMARY

An object of this invention is to provide a fatigue life prediction method of concrete with concise steps, simpleness to use and high precision. To this end, the present invention employs the following technical solutions.

A fatigue life prediction method of concrete based on Weibull function and maximum fatigue deformation, comprising the following steps:

(1) acquire several (i) maximum fatigue deformation εₛ and the number of fatigue load cycles n corresponding to each deformation of the concrete under a fatigue load at one certain stress level, i.e. (εₛ₁, n₁), (εₛ₂, n₂), (εₛ₃, n₃), . . . , (εₛᵢ, . . . , nᵢ); the maximum fatigue deformation e, refers to the deformation corresponding to the maximum stress in a cycle of fatigue load;

(2) substitute the several (i) maximum fatigue deformations and the corresponding fatigue life cycle into the following equation for fitting and solving, to obtain parameters of the equation:

n=N_{f}(1−exp(−((εₛ−εₛ₀)/λₛ)^{k}^{ₛ}))

wherein, N_{f} is fatigue life, εₚ₀ is position parameter, λₚ is scale parameter, kₚ is shape parameter.

The parameter N_{f} obtained in step (2) is the prediction of fatigue life, and the resulting equation is used to characterize the evolution law of fatigue deformation.

Further, an optional value for position parameter 6, is the deformation corresponding to the maximum stress of the first fatigue load cycle of the concrete.

Further, λₛ/kₛ is set to the same value for the same kind of concrete material. Further, the same value can be the strain rate of the second stage in the fatigue deformation versus normalized fatigue life curve of the concrete material, i.e. ∂^{ε}/∂(n/N_{f}), so as to simplify the fitting process and improve the accuracy of the prediction results.

Another object of the invention is to provide a fatigue life prediction device of concrete based on Weibull function and maximum fatigue deformation, to this end, the present invention employs the following technical solutions.

A fatigue life prediction device of concrete based on Weibull function and maximum fatigue deformation, comprising a data acquisition module, a parameter determination module and an information transmission module;

The data acquisition module is used to acquire several maximum fatigue deformation e, and the number of fatigue load cycles n corresponding to each deformation of the concrete under a fatigue load at one certain stress level; the maximum fatigue deformation εₛ refers to the deformation corresponding to the maximum stress in a cycle of fatigue load;

The parameter determination module is used to substitute the several maximum fatigue deformations and the corresponding fatigue life cycle into the following equation for fitting and solving, to obtain parameters of the equation:

n=N_{f}(1−exp(−((εₛ−εₛ₀)/λₛ)^{k}^{ₛ}))

wherein, N_{f} is fatigue life, εₛ₀ is position parameter, λₛ is scale parameter, kₛ is shape parameter.

The information transmission module is used to transmit parameters of the equation obtained by fitting and solving to a fixed receiver or a mobile receiver, wherein the parameter includes N_{f}.

The invention provides a fatigue life prediction method and device of concrete based on Weibull function and maximum fatigue deformation. Using the method and device, as long as acquiring several maximum fatigue deformations εₛ and the number of fatigue load cycles n corresponding to each deformation, and substituting them into the equation for fitting and solving, the fatigue life and deformation evolution law can be obtained, having the advantages of concise steps, simpleness to use and high accuracy, etc. In the process of using, it can greatly reduce the calculation, and only need to measure two fatigue parameters of the number of fatigue load cycles n and the maximum fatigue deformation εₛ of the n^{th} cycle, which can simplify the monitoring equipment. The model can provide important technical support for engineering design, construction, monitoring and maintenance.

### BRIEF DESCRIPTION OF THE DRAWINGS

The sole FIGURE is a graph of actual measured results and predicted results of the maximum deformation and fatigue life of fiber-reinforced concrete under the fatigue load according to Example 1 of the present invention.

### DETAILED DESCRIPTION

The present invention is further described in combination with drawings and specific embodiments. The embodiments are intended to illustrate the present invention, but not to limit the invention in any way.

This example predicts the compression fatigue life and characterizes the evolution law of fatigue deformation of three fiber concrete samples with the stress levels of 0.85, 0.80, and 0.75 respectively.

For the same concrete material, λₛ/kₛ can be set to the same value. Therefore, in this example, a compressive fatigue test on three samples of the same type of fiber-reinforced concrete at a stress level of 0.90 is performed firstly, to obtain the average value of λₛ/kₛ as the same value set as described. Through the compression fatigue test, 15 maximum fatigue deformations εₛ and the corresponding number of fatigue load cycles n of these three samples (as shown in Table 1) are obtained. Moreover, the fatigue life N_{f} of these three samples are measured.

The maximum fatigue deformation of each sample and the corresponding fatigue load cycle are substituted into the following equation for fitting and solving, to get the parameters of the equation.

n=N_{f}(1−exp(−((εₛ−εₛ₀)/λₛ)^{k}^{ₛ}))

Wherein, the fitting values of position parameter εₛ₀, scale parameter λₛ, and shape parameter kₛ are shown in Table 1. The average value of λₛ/kₛ of samples 1, 2, and 3 is 0.06815.

TABLE 1
The compressive fatigue data of fiber-reinforced concrete samples at for a stress level of 0.90

| Number of | | Number of | | Number of | |
|---|---|---|---|---|---|
| fatigue load | Maximum fatigue | fatigue load | Maximum fatigue | fatigue load | Maximum fatigue |
| cycles of | deformations | cycles of | deformations | cycles of | deformations |
| sample 1, n | of sample 1, εₛ/% | sample 2, n | of sample 2, εₛ/% | sample 3, n | of sample 3, εₛ/% |
| 1 | 0.5143 | 1 | 0.5017 | 1 | 0.5403 |
| 11 | 0.5446 | 7 | 0.5240 | 19 | 0.5879 |
| 55 | 0.5940 | 35 | 0.5654 | 95 | 0.6409 |
| 110 | 0.6380 | 70 | 0.5921 | 190 | 0.6765 |
| 220 | 0.6576 | 140 | 0.6412 | 380 | 0.7093 |
| 330 | 0.6769 | 210 | 0.6634 | 570 | 0.7283 |
| 440 | 0.6971 | 280 | 0.6854 | 760 | 0.7422 |
| 550 | 0.7139 | 350 | 0.7102 | 950 | 0.7599 |
| 660 | 0.7254 | 420 | 0.7296 | 1140 | 0.7753 |
| 770 | 0.7435 | 490 | 0.7516 | 1330 | 0.7959 |
| 880 | 0.7579 | 560 | 0.7768 | 1520 | 0.8234 |
| 990 | 0.7897 | 630 | 0.8123 | 1710 | 0.8659 |
| 1045 | 0.8099 | 665 | 0.8316 | 1805 | 0.8968 |
| 1095 | 0.8664 | 697 | 0.8550 | 1891 | 0.9396 |
| 1099 | 0.9125 | 699 | 0.8613 | 1899 | 0.9522 |

| | | |
|---|---|---|
| N_{f1} = 1100 (measured) | N_{f2} = 700 (measured) | N_{f3} = 1900 (measured) |
| kₛ₁ = 4.28069 (fitting) | kₛ₂ = 4.72057 (fitting) | kₛ₃ = 3.49126 (fitting) |
| λₛ₁ = 0.24869 (fitting) | λₛ₂ = 0.36632 (fitting) | λₛ₃ = 0.24004 (fitting) |
| εₛ₀₁ = 0.48347 (fitting) | εₛ₀₂ = 0.37024 (fitting) | εₛ₀₃ = 0.54030 (fitting) |
| λₛ₁/kₛ₁ = 0.05810 | λₛ₂/kₛ₂ = 0.07760 | λₛ₃/kₛ₃ = 0.06875 |

Next, the prediction of the compressive fatigue life and characterization of the evolution law of fatigue deformation are performed for the three fiber-reinforced concrete samples at the stress levels of 0.85, 0.80 and 0.75, respectively.

(1) acquire 9 maximum fatigue deformation εₛ and the number of fatigue load cycles n corresponding to each deformation of the fiber-reinforced concrete under a fatigue load at the stress levels of 0.85, 0.80 and 0.75, respectively (as shown in table 2);

(2) substitute these 9 maximum fatigue deformations and the corresponding fatigue life cycle into the following equation for fitting and resolving, to obtain parameters of the equation:

n=N_{f}(1−exp(−((εₛ−εₛ₀)/λₛ)^{k}^{ₛ}))

It should be noted that in the fitting solution process, the value of λₛ/kₛ of the fiber-reinforced concrete is set at 0.06815.

The fitted values of the fatigue life N_{f}, position parameter εₛ₀, scale parameter λₛ, and shape parameter kₛ at various stress levels obtained by fitting and solving are shown in Table 2. The actual valves of fatigue life N_{f} at various stress levels are also shown in Table 2. It can be found that the predicted value obtained by the fitting is close to the actual value and the prediction accuracy is high. The obtained test data of each sample in Table 2 and the equation of fitting solution are shown in the sole FIGURE. Further, the subsequent fatigue data that is not obtained during the fitting solution is also plotted in the sole FIGURE. It can be found that there is a strong correlation between the fitting results and the prediction results based on the equation.

TABLE 2
The compressive fatigue data of fiber-reinforced concrete
samples at the stress levels of 0.85, 0.80 and 0.75

| Number of | | Number of | | Number of | |
|---|---|---|---|---|---|
| fatigue load | Maximum fatigue | fatigue load | Maximum fatigue | fatigue load | Maximum fatigue |
| cycles of | deformations | cycles of | deformations | cycles of | deformations |
| sample at | of sample at the | sample at | of sample at the | samples at | of sample at the |
| stress level | stress level | stress level | stress level | stress level | stress level |
| of 0.85, n | of 0.85, εₛ/% | of 0.80, n | of 0.80, εₛ/% | of 0.75, n | of 0.75, εₛ/% |
| 1 | 0.4681 | 1 | 0.4532 | 1 | 0.4031 |
| 41 | 0.5433 | 359 | 0.5456 | 7183 | 0.5280 |
| 206 | 0.5997 | 1795 | 0.5897 | 35917 | 0.5663 |
| 412 | 0.6259 | 3591 | 0.6133 | 71834 | 0.5895 |
| 823 | 0.6532 | 7182 | 0.6353 | 143669 | 0.6158 |
| 1235 | 0.6692 | 10772 | 0.6552 | 215503 | 0.6431 |
| 1646 | 0.6880 | 14363 | 0.6707 | 287337 | 0.6596 |
| 2058 | 0.7095 | 17954 | 0.6919 | 359172 | 0.6768 |
| 2469 | 0.7252 | 21545 | 0.7190 | 431006 | 0.6945 |

| | | |
|---|---|---|
| k_{s-0.85} = 3.9946 (fitting) | k_{s-0.80} = 3.8438 (fitting) | k_{s-0.75} = 4.5463 (fitting) |
| λ_{s-0.85} = 0.2722 (fitting) | λ_{s-0.80} = 0.2620 (fitting) | λ_{s-0.75} = 0.3098 (fitting) |
| ε_{s0-0.85} = 0.4681 (fitting) | ε_{s0-0.80} = 0.4532 (fitting) | ε_{s0-0.75} = 0.4031 (fitting) |
| N_{f-0.85} = 4540 (fitting) | N_{f-0.80} = 34331 (fitting) | N_{f-0.75} = 821648 (fitting) |
| N_{f-0.85} = 4115 (actual life) | N_{f-0.80} = 35908 (actual life) | N_{f-0.75} = 718343 (actual life) |

What is claimed is:

1. A fatigue life prediction method of concrete based on Weibull function and maximum fatigue deformation, comprising the following steps:
(1) acquire several maximum fatigue deformations εₛ and the number of fatigue load cycles n corresponding to each deformation of the concrete under a fatigue load at one certain stress level; the maximum fatigue deformation εₛ refers to the deformation corresponding to the maximum stress in a cycle of fatigue load;
(2) substitute the several maximum fatigue deformations and corresponding fatigue load cycles into the following equation for fitting and solving, to obtain parameters of the equation:
n=N_{f}(1−exp(−((εₛ−εₛ₀)/λₛ)^{k}^{ₛ}))
wherein, N_{f} is fatigue life, εₚ₀ is position parameter, λₚ is scale parameter, kₚ is shape parameter;
The parameter N_{f} obtained in step (2) is the prediction of fatigue life, and the resulting equation is used to characterize the evolution law of fatigue deformation.

2. The fatigue life prediction method of concrete based on Weibull function and maximum fatigue deformation according to claim 1, wherein an optional value for position parameter εₛ₀ is the deformation corresponding to the maximum stress of the first fatigue load cycle of the concrete.

3. The fatigue life prediction method of concrete based on Weibull function and maximum fatigue deformation according to claim 1, wherein λₛ/kₛ is set to the same value for the same kind of concrete material.

4. A fatigue life prediction device of concrete based on Weibull function and maximum fatigue deformation, comprising a data acquisition module, a parameter determination module and an information transmission module;
The data acquisition module is used to acquire several maximum fatigue deformations εₛ and the number of fatigue load cycles n corresponding to each deformation of the concrete under a fatigue load at one certain stress level; the maximum fatigue deformation εₛ refers to the deformation corresponding to the maximum stress in a cycle of fatigue load;
The parameter determination module is used to substitute the several maximum fatigue deformations and the corresponding fatigue life cycles into the following equation for fitting and solving, to obtain parameters of the equation:
n=N_{f}(1−exp(−((εₛ−εₛ₀)/λₛ)^{k}^{ₛ}))
wherein, N_{f} is fatigue life, εₛ₀ is position parameter, λₛ is scale parameter, kₛ is shape parameter;
The information transmission module is used to transmit parameters of the equation obtained by fitting and solving to a fixed receiver or a mobile receiver, wherein the parameter includes N_{f}.
